(19) United States
(12) Patent Application Publication
(10) Pub. No.: US 2018/0116634 A1
(43) Pub. Date: May 3, 2018

(54) ULTRASONIC DIAGNOSIS APPARATUS

(71) Applicant: Seiko Epson Corporation, Tokyo (JP)

(72) Inventor: Tomonori MANO, Fujimi (JP)

(21) Appl. No.: 15/724,590

(22) Filed: Oct. 4, 2017

(30) Foreign Application Priority Data
Oct. 31, 2016 (JP) 2016-212647

(51) Int. Cl. A61B 8/00; A61B 8/14; A61B 8/08

(52) U.S. Cl. CPC A61B 8/467; A61B 8/461; G06F 3/044; A61B 8/14; A61B 8/488; A61B 8/54

(57) ABSTRACT

An ultrasonic diagnosis apparatus includes an ultrasonic element array that has two-dimensionally arranged ultrasonic elements transmitting ultrasonic waves and receiving reflected echoes, an ultrasonic image generator that generates an ultrasonic image on the basis of the reflected echoes received by the ultrasonic element array, a display that displays the ultrasonic image, a touch panel that is provided on a display surface of the display and receives a user's input so as to output an input signal, and a driving controller that controls driving of the ultrasonic element array on the basis of the input signal from the touch panel.

### BACKGROUND

### 1. Technical Field

The present invention relates to an ultrasonic diagnosis apparatus.

### 2. Related Art

In a diagnosis method using an ultrasonic diagnosis apparatus, two-dimensional image data can be observed in real time only with a simple operation of bringing an ultrasonic probe into contact with a body surface, and thus the diagnosis method is widely used for function diagnosis or morphology diagnosis for a living body organ.

In recent years, a method has been developed in which three-dimensional image data is generated through mechanical movement of an ultrasonic probe in which vibrating elements are arranged in a one-dimensional manner, or by using a so-called two-dimensional array ultrasonic probe in which vibrating elements are arranged in a two-dimensional manner. A method has also been proposed in which two-dimensional image data and three-dimensional image data of a diagnosis target part collected by using the same ultrasonic probe are combined and displayed. Particularly, JP-A-2008-188287 discloses an ultrasonic diagnosis apparatus using an ultrasonic probe in which vibrating elements are arranged in a two-dimensional manner. According to the disclosure, it is possible to observe two-dimensional image data in which a spatial resolution and a temporal resolution are excellent and three-dimensional image data enabling wide range observation in real time without moving the ultrasonic probe disposed on a body surface of a subject.

Miniaturization of an ultrasonic diagnosis apparatus has progressed, and a portable ultrasonic diagnosis apparatus has been spread. In a case of a portable ultrasonic diagnosis apparatus, a touch screen is useful as a principal user interface, and such a button or a track ball provided in a diagnosis apparatus of the related art is not necessary. A touch operation includes operation methods such as pinch and swipe, which may be allocated with enlargement and reduction of a screen, and gain or depth adjustment.

According to WO2013/132747, a piezoelectric device used in an ultrasonic probe tends to be miniaturized and thinned, and this can be realized by using a semiconductor process. If such a thin piezoelectric device is used, an ultrasonic probe is miniaturized and thinned. A handy type probe is used for ultrasonic diagnosis in the related art, but more versatile probes can be created so as to be used for diagnosis.

In a case where a small-sized and thin two-dimensional array ultrasonic probe is realized, a device which fixes the probe around a measurement part in order to perform observation is considered. A user can concentrate on image observation without contacting the probe fixed around the measurement part, and the device may be used for normal monitoring or observation on emergency. Features of the two-dimensional array ultrasonic probe are that three-dimensional volume data can be acquired, and any section can be observed even in a typical two-dimensional image. It is possible to observe any section without contacting the probe due to these features.

However, in the apparatus of the related art disclosed in JP-A-2008-188287, conditions for generation of two-dimensional image data or generation of three-dimensional image data are set by using a keyboard, various switches and buttons, a track ball, and the like, and thus there is a problem in that the setting is cumbersome, and, in a case where various sections are desired to be observed in real time, it is not possible to easily perform observation. Even in a case where a measurement section desired to be observed is selected, there may be a method in which any section is selected on the basis of preserved three-dimensional volume data in the related art, but the data is not real time data, and thus deviation or distortion occurs in an observation part so that it is difficult to accurately select a measurement section. Therefore, there is the need for an ultrasonic diagnosis apparatus which can easily perform real time driving control based on a user's input.

### SUMMARY

An advantage of some aspects of the invention is to solve the problem described above, and the invention can be implemented as the following forms or application examples.

### APPLICATION EXAMPLE 1

An ultrasonic diagnosis apparatus according to this application example includes an ultrasonic element array that has two-dimensionally arranged ultrasonic elements transmitting ultrasonic waves and receiving reflected echoes; an ultrasonic image generator that generates an ultrasonic image on the basis of the reflected echoes received by the ultrasonic element array; a display that displays the ultrasonic image; a touch panel that is provided on a display surface of the display and receives a user's input so as to output an input signal; and a driving controller that controls driving of the ultrasonic element array on the basis of the input signal from the touch panel.

According to this application example, the ultrasonic element array having two-dimensional arrangement transmits ultrasonic waves and receives reflected echoes. The ultrasonic image generator generates an ultrasonic image on the basis of the received reflected echoes. The display displays the generated ultrasonic image. A user performs a touch operation on the touch panel provided on the display surface of the display. The driving controller which controls driving of the ultrasonic element array controls driving of the ultrasonic element array on the basis of an input signal from the touch panel due to a touch operation. The touch operation is easier than a keyboard operation or a track ball operation. Therefore, the user can easily perform real-time driving control based on the user's touch operation.

### APPLICATION EXAMPLE 2

In the ultrasonic diagnosis apparatus according to the application example, it is preferable that the driving controller performs driving control according to any of the type of touch operation, the velocity of the operation, and a movement amount of the operation which are input from the touch panel.

According to this application example, the driving controller changes driving control according to the type of touch operation, the velocity of the operation, and a movement amount of the operation which are input from the touch panel. Thus, a user can easily change driving control for the ultrasonic element array. The type of touch operation includes touch of bringing the finger into contact with a screen, tap of tapping a screen, swipe of sliding the finger in a touched state, and pinch of pinching a screen with two fingers.

### APPLICATION EXAMPLE 3

In the ultrasonic diagnosis apparatus according to the application example, it is preferable that the driving controller controls either one of a driving scanning surface and a scanning surface angle in the ultrasonic element array on the basis of the input signal from the touch panel.

According to this application example, the driving controller can easily switch between observation regions by controlling a driving scanning surface in the ultrasonic element array having two-dimensional arrangement. Alternatively, it is possible to easily switch between observation angles by controlling a scanning surface angle.

### APPLICATION EXAMPLE 4

It is preferable that the ultrasonic diagnosis apparatus according to the application example further includes a storage that stores initial driving conditions, and the driving controller performs driving control under the initial driving conditions on the basis of input from the touch panel.

According to this application example, the driving controller performs driving control on the basis of the initial driving conditions stored in advance and input from the touch panel. Thus, a user can easily execute the initial driving conditions even after driving conditions are variously changed.

### APPLICATION EXAMPLE 5

In the ultrasonic diagnosis apparatus according to the application example, it is preferable that the driving controller performs driving in a high resolution mode in a case where there is no input from the touch panel, and performs driving in a low resolution mode in a case where there is input from the touch panel, and the driving is performed so that the number of scanning lines is reduced or a scanning line interval is increased in the low resolution mode more than in the high resolution mode.

According to this application example, the driving controller controls driving of the ultrasonic element array in the high resolution mode in a case where there is no input from the touch panel. The driving controller controls driving of the ultrasonic element array in the low resolution mode in a case where there is input from the touch panel. The number of scanning lines is reduced or a scanning line interval is increased in the low resolution mode more than in the high resolution mode. Thus, in a case where there is no input from the touch panel, a user can observe a high-resolution image in the high resolution mode. In a case where there is input from the touch panel, the user can observe an image in the low resolution mode without image display delay. The case where there is no input from the touch panel indicates a state in which a user does not contact the touch panel, or there is no change of a threshold value or greater in a position or the intensity of an input signal from the touch panel. The case where there is input from the touch panel indicates a state in which there is a change of a threshold value or greater in a position or the intensity of an input signal from the touch panel.

### BRIEF DESCRIPTION OF THE DRAWINGS

The invention will be described with reference to the accompanying drawings, wherein like numbers reference like elements.

FIG. 1 is a configuration diagram of an ultrasonic diagnosis apparatus according to Embodiment 1.

FIG. 2 is a functional block diagram of the ultrasonic diagnosis apparatus.

FIG. 3A is a diagram for explaining an ultrasonic beam scanning direction.

FIG. 3B is a diagram for explaining an ultrasonic beam scanning direction.

FIG. 3C is a diagram for explaining an ultrasonic beam scanning direction.

FIG. 4 is a conceptual diagram illustrating a method of detecting input on a projection capacitance type touch panel.

FIG. 5 is a diagram for explaining measurement of a speed of an operation on the touch panel and a movement amount thereof.

FIG. 6 is a flowchart illustrating an operation of the ultrasonic diagnosis apparatus.

FIG. 7A is a diagram illustrating a B mode image on a driving scanning surface under initial driving conditions.

FIG. 7B is a diagram for explaining a two-dimensional scanning range corresponding to the B mode image in FIG. 7A.

FIG. 8A is a diagram illustrating an operation on the touch panel and a B mode image when z-axis rotation driving control is performed.

FIG. 8B is a diagram for explaining a two-dimensional scanning range corresponding to the operation on the touch panel and the B mode image in FIG. 8A.

FIG. 9A is a diagram illustrating an operation on the touch panel and a B mode image when y-axis rotation driving control is performed.

FIG. 9B is a diagram for explaining a two-dimensional scanning range corresponding to the operation on the touch panel and the B mode image in FIG. 9A.

FIG. 10A is a diagram illustrating an operation on the touch panel and a B mode image when x-axis rotation driving control is performed.

FIG. 10B is a diagram for explaining a two-dimensional scanning range corresponding to the operation on the touch panel and the B mode image in FIG. 10A.

FIG. 11A is a diagram illustrating an operation on the touch panel and a B mode image when driving control is performed under initial driving conditions.

FIG. 11B is a diagram for explaining a two-dimensional scanning range corresponding to the operation on the touch panel and the B mode image in FIG. 11A.

FIG. 12A is a diagram illustrating an operation on the touch panel and a B mode image when enlargement/reduction driving control is performed.

FIG. 12B is a diagram for explaining a two-dimensional scanning range corresponding to the operation on the touch panel and the B mode image in FIG. 12A.

FIG. 13 a diagram illustrating driving condition correspondence.

FIG. 14A is a diagram illustrating an operation on the touch panel and a B mode image when rotation angle control is performed.

FIG. 14B is a diagram for explaining a two-dimensional scanning range corresponding to the operation on the touch panel and the B mode image in FIG. 14A.

FIG. 15A is a diagram illustrating an operation on the touch panel and a B mode image when rotation angle control is performed with respect to a case where a movement amount of a touch operation is larger than that in FIG. 14A.

FIG. 15B is a diagram for explaining a two-dimensional scanning range corresponding to the operation on the touch panel and the B mode image in FIG. 15A.

FIG. 16 is a diagram for explaining a relationship between a movement amount of a touch operation and a rotation angle of a scanning range.

FIG. 17 is a diagram for explaining a relationship between a movement amount of a touch operation and a rotation angle of a scanning range.

FIG. 18A is a diagram illustrating a B mode image on a driving scanning surface under initial driving conditions.

FIG. 18B is a diagram for explaining a two-dimensional scanning range corresponding to the B mode image in FIG. 18A.

FIG. 18C is a schematic diagram illustrating ultrasonic beams in the scanning range in FIG. 18B as arrows.

FIG. 19A is a diagram illustrating a B mode image obtained through an operation on the touch panel in a low resolution mode.

FIG. 19B is a diagram for explaining a two-dimensional scanning range corresponding to the B mode image in FIG. 19A.

FIG. 19C is a schematic diagram illustrating ultrasonic beams in the scanning range in FIG. 19B as arrows.

FIG. 20A is a diagram illustrating a B mode image obtained through an operation on the touch panel in a narrow visual field mode.

FIG. 20B is a diagram for explaining a two-dimensional scanning range corresponding to the B mode image in FIG. 20A.

FIG. 20C is a schematic diagram illustrating ultrasonic beams in the scanning range in FIG. 20B as arrows.

FIG. 21 is a diagram for explaining display of a screen regarding a relationship among a B mode image in a two-dimensional scanning range, an ultrasonic element array, and an ultrasonic beam scanning surface.

### DESCRIPTION OF EXEMPLARY EMBODIMENTS

Hereinafter, an embodiment of the invention will be described with reference to the drawings. In the following diagrams, a scale of each layer or each member is different from actual one since each layer or each member is exaggerated to be able to be recognized.

### Embodiment 1

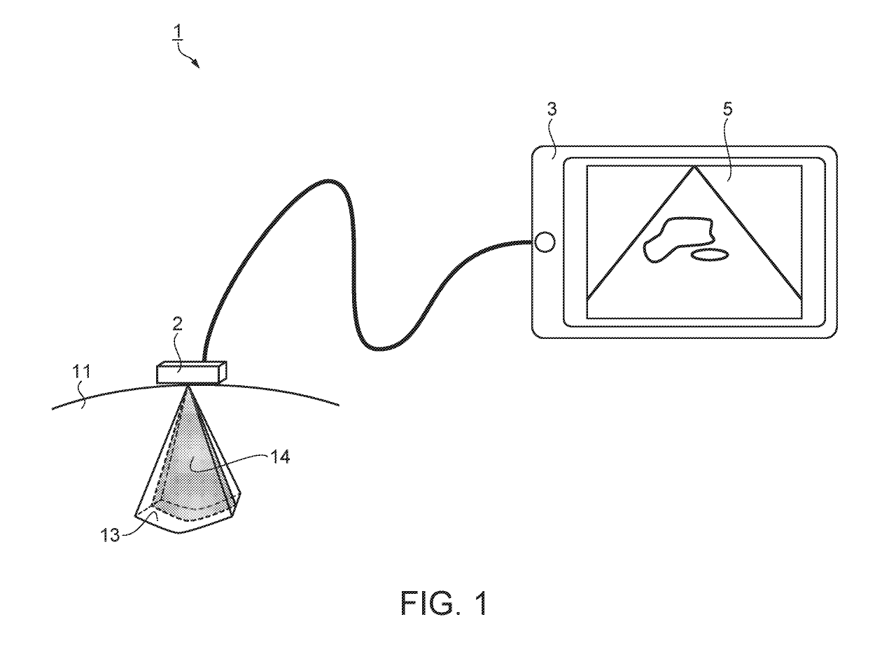

FIG. 1 is a configuration diagram of an ultrasonic diagnosis apparatus. First, with reference to FIG. 1, a description will be made of a configuration of an ultrasonic diagnosis apparatus according to Embodiment 1.

An ultrasonic diagnosis apparatus 1 obtains a two-dimensional or three-dimensional tomographic image of the inside of a subject 11 by bringing an ultrasonic probe 2 into contact with the subject 11. A user (not illustrated) performs ultrasonic wave driving control by using an ultrasonic diagnosis apparatus main body 3 including a display 5. The subject mentioned here indicates an animal such as a person, a dog, a cat, a cow, or a horse. The ultrasonic diagnosis apparatus 1 may also be used for fish, plants, or metal.

The ultrasonic probe 2 transmits ultrasonic waves to the subject 11 via ultrasonic gel or water (not illustrated), and receives a reflected echo. The ultrasonic waves are transmitted and received within a three-dimensional volume scanning range 13 or a two-dimensional scanning range 14. Consequently, a two-dimensional or three-dimensional tomographic image of the inside of the subject 11 can be acquired.

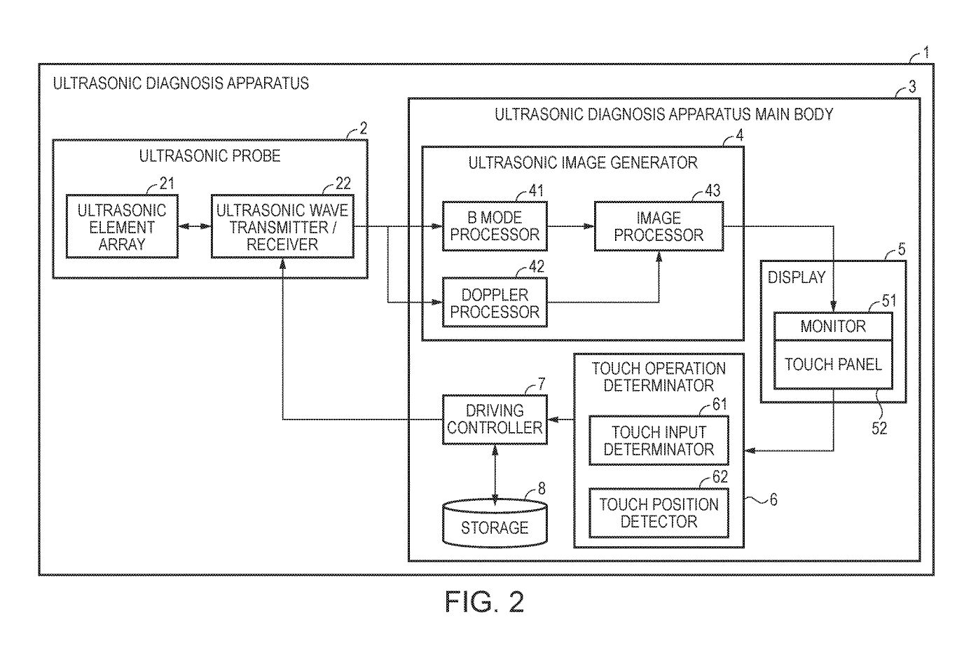

FIG. 2 is a functional block diagram of the ultrasonic diagnosis apparatus. With reference to FIG. 2, a description will be made of a function of the ultrasonic diagnosis apparatus.

The ultrasonic diagnosis apparatus 1 includes an ultrasonic element array 21 which transmits ultrasonic pulses to the subject 11, and converts a received reflected echo into an electrical signal. The ultrasonic element array 21 is an array of two-dimensionally arranged ultrasonic elements which are mounted on the ultrasonic probe 2, transmit ultrasonic waves, and receive reflected echoes. An ultrasonic wave transmitter/receiver 22 is provided to be connected to the ultrasonic element array 21. The ultrasonic wave transmitter/receiver 22 supplies a driving signal for transmitting ultrasonic pulses in a predetermined direction of the subject 11, to the ultrasonic element array 21, receives reflected echoes, and performs a phasing addition process on received signals. An ultrasonic image generator 4 is provided to be connected to the ultrasonic wave transmitter/receiver 22. The ultrasonic image generator 4 generates an ultrasonic image on the basis of a reflected echo received by the ultrasonic element array 21. The ultrasonic image generator 4 includes a B mode processor 41, a Doppler processor 42, and an image processor 43. The B mode processor 41 and the Doppler processor 42 are connected to the ultrasonic wave transmitter/receiver 22, and are also connected to the image processor 43. The image processor 43 is connected to the display 5. The B mode processor 41 performs a brightness (B) mode process such as an envelope process or logarithmic conversion on received signals having undergone a phasing addition process. The Doppler processor 42 performs a Doppler mode signal process of calculating a phase difference between frames of the received signals having undergone the phasing addition process so as to acquire blood flow information or the like. The image processor 43 included in the ultrasonic image generator 4 performs predetermined image conversion or image processing on the basis of a signal having undergone the B mode process or the Doppler mode process, so as to generate an ultrasonic image.

The display 5 is provided to be connected to the ultrasonic image generator 4. The display 5 includes a monitor 51 and a touch panel 52. The display 5 displays ultrasonic image data as a generated ultrasonic image or driving conditions for the ultrasonic wave transmitter/receiver 22 on the monitor 51. The touch panel 52 for the user performing a touch operation is provided on a display surface of the display 5. The touch panel 52 receives the user's input and outputs an input signal. A touch operation determinator 6 is provided to be connected to the display 5. The touch operation determinator 6 includes a touch input determinator 61 and a touch position detector 62. The touch input determinator 61 determines whether or not the user starts touch input. The touch position detector 62 detects a position of a touch operation performed by the user. The touch operation determinator 6 determines the type of touch operation performed by the user, an operation speed, a movement amount of an operation, or the like. A driving controller 7 is provided to be connected to the touch operation determinator 6. The driving controller 7 performs driving control on the ultrasonic element array 21 on the basis of an input signal from the touch panel 52, generated by touch operation input. The driving controller 7 is implemented by, for example, a microcomputer such as a CPU or a graphic processor unit (GPU), or electric components such as an ASIC, a field-programmable gate array (FPGA), an integrated circuit (IC), and a memory. A storage 8 is provided to be connected to the driving controller 7. Information such as initial driving conditions is stored in the storage 8. The driving controller 7 is connected to the ultrasonic wave transmitter/receiver 22, and outputs an instruction signal or the like for driving the ultrasonic elements to the ultrasonic wave transmitter/receiver 22. The ultrasonic diagnosis apparatus 1 has such functions.

A driving signal for transmitting an ultrasonic pulse is a signal obtained by adding a deflection delay time for deflection in any direction and a focusing delay time for focusing in any depth. The phasing addition process on received signals is a process in which a deflection delay time for enabling a signal from any direction to have strong reception directivity and a focusing delay time for focusing a signal from any depth are applied to the received signals from the respective ultrasonic elements, and the received signals are added together.

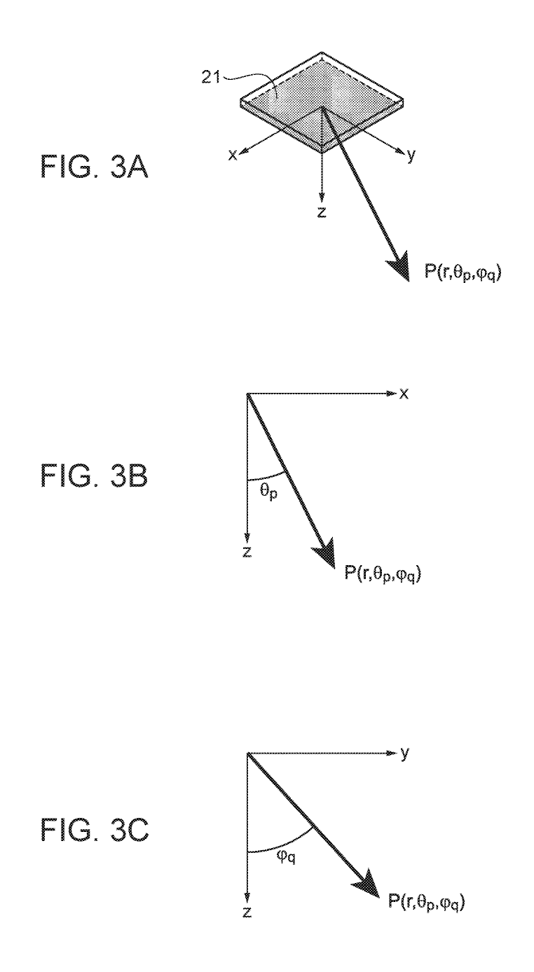

FIGS. 3A, 3B and 3C are diagrams for explaining an ultrasonic beam scanning direction. With reference to FIGS. 3A, 3B and 3C, a description will be made of an ultrasonic beam scanning direction with a central axis of the ultrasonic element array 21 having two-dimensional arrangement as a z axis. A sensor surface of the ultrasonic element array 21 is located on an xy plane, and the sensor surface is directed in a z axis direction. An ultrasonic beam transmitted from the ultrasonic element array 21 is irradiated in any direction (θp, θq). In a case of projection onto an xz plane and a yz plane, an angle from the z axis in the xz plane is θp, and an angle from the z axis in the yz plane is θq. The ultrasonic element array 21 has N ultrasonic elements (not illustrated) which are arranged in a two-dimensional manner. The N ultrasonic elements arranged in a two-dimensional manner are arranged on the xy plane, and can be driven separately. Consequently, the ultrasonic element array 21 having the two-dimensional arrangement can transmit and receive ultrasonic waves in any direction.

The ultrasonic probe 2 includes a sector scanning type, a linear scanning type, a convex scanning type, and the like, and may be selected by the user according to a diagnosis part. In the present embodiment, a case where the ultrasonic probe 2 is of the sector scanning type will be described, but other scanning types may be used.

Referring to FIG. 2 again, image processing performed by the ultrasonic image generator 4 includes, for example, a so-called scan conversion process of synthesizing a two-dimensional image by performing an interpolation process on signals from the respective ultrasonic elements, and a volume rendering process of synthesizing a three-dimensional image. The image processing also includes a process of combining images processed in a B mode and a Doppler mode into a single image. The image processing also includes a process of generating image data at a time phase which cannot be acquired, through an interpolation process. Such image data maybe immediately displayed on the display 5, and may be stored in a memory (not illustrated) which temporarily preserves images so as to be displayed on the display 5 in the future.

As the monitor 51, a monitor employing a liquid crystal display (LCD) or an organic light emitting display (OLED) may be used. As the touch panel 52, a projection capacitance type touch panel or a resistance film type touch panel may be used. The projection capacitance type is a type of detecting a change in capacitance when a touch surface to which a transparent electrode film is bonded is touched. The resistance film type is a type of detecting a resistance change caused by contact between upper and lower electrode films due to pressing when a touch surface formed of two-layered transparent electrode films such as upper and lower films is touched. However, the projection capacitance type enables accurate multi-point detection (multi-touch), and is thus suitable for Embodiment 1.

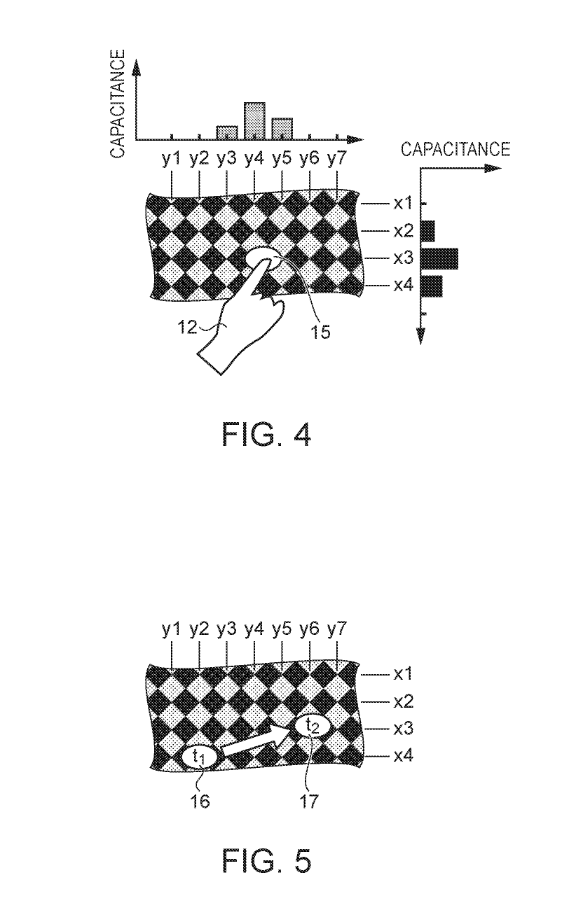

FIG. 4 is a conceptual diagram illustrating a method of detecting input on a projection capacitance type touch panel. As illustrated in FIG. 4, transparent electrodes are arranged in a matrix at respective positions of X coordinates x1 to x4 and Y coordinates y1 to y7. If the finger of a user 12 comes close to a position 15 in the figure, the capacitance of the transparent electrode changes. In this case, since a capacitance change of the transparent electrode located at the X coordinate of x3 and the Y coordinate of y4 is greatest, the user 12 can understand that coordinates of the touched position are (x3, y4). Alternatively, position detection with high accuracy may be performed on the basis of a proportion of a capacitance change.

FIG. 5 is a diagram for explaining measurement of a speed of an operation on the touch panel and a movement amount thereof. With reference to FIG. 5, a description will be made of a case of performing an operation (one-finger swipe) of sliding the finger from a position 16 to a position 17 in the figure. The touch position detector 62 detects the position 16 (x4, y2) on the basis of an input signal from the touch panel at a time point t1. Next, the touch position detector 62 detects the position 17 (x3,y6) on the basis of an input signal from the touch panel at a time point t2. The velocity v of a touch operation can be calculated according to Equation (1). A movement amount d of a touch operation can be calculated according to Equation (2).

$$v = \frac{\sqrt{{( {x}_{3} - {x}_{4} )}^{2} + {( {y}_{6} - {y}_{2} )}^{2}}}{{t}_{2} - {t}_{1}} \qquad ( 1 )$$

d=\sqrt{(x₃−x₄)²+(y₆−y₂)²} (2)

The types of touch operations include touch of bringing the finger into contact with a touch surface of the touch panel 52, tap of tapping the touch surface, swipe (or also referred to as flick) of sliding the finger in a touched state, and pinch of pinching or unpinching the touch surface with two fingers.

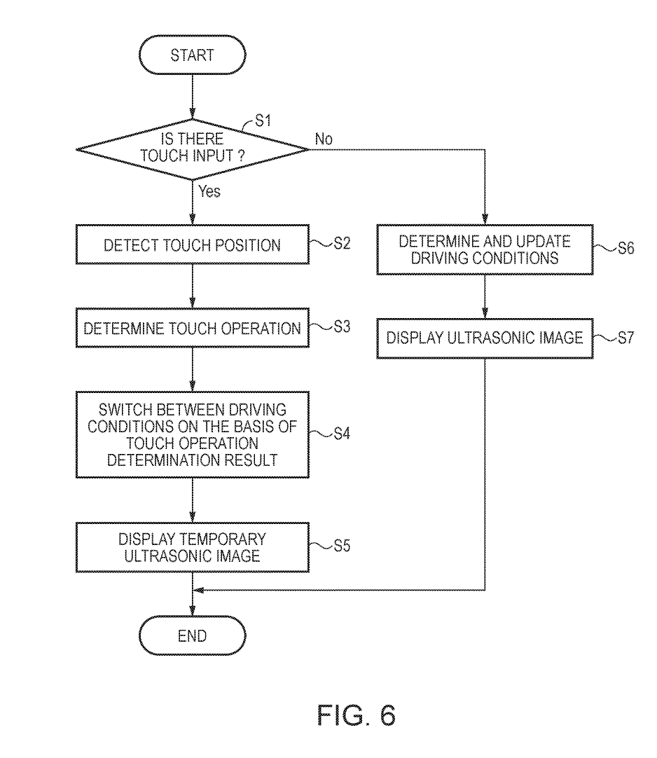

FIG. 6 is a flowchart illustrating an operation of the ultrasonic diagnosis apparatus 1. A description will be made of an operation flow with reference to FIG. 6.

The user performs touch input by using the touch panel 52 of the ultrasonic diagnosis apparatus 1. The touch input determinator 61 detects the presence or absence of touch input (step S1 in FIG. 6). For example, the touch input determinator 61 determines the presence or absence of a capacitance change of the touch panel 52 through comparison with a determination value, so as to detect touch input.

In a case where there is touch input (Yes in step S1), the touch position detector 62 detects a touch position every predetermined sampling time (step S2 in FIG. 6). The touch operation determinator 6 calculates the type of touch operation, the velocity of the operation, and a movement amount of the operation on the basis of a touch position result every sampling time which is output information from the touch position detector 62, so as to determine a touch operation (step S3 in FIG. 6).

The touch operation determinator 6 inputs a touch operation determination result to the driving controller 7. The driving controller 7 controls driving of the ultrasonic wave transmitter/receiver 22 through switching to predetermined driving conditions on the basis of the touch operation determination result. In this case, the driving conditions are selected and determined from a correspondence table (lookup table: LUT) in which touch operation determination results and driving conditions, or methods of changing driving conditions are set in advance. The storage 8 holds the LUT in a memory or the like in advance. The driving controller 7 may read the LUT from the storage 8 at suitable time (step S4 in FIG. 6).

In a case where there is touch input, transmission and reception of ultrasonic waves are performed with the predetermined driving conditions as temporary driving conditions, and a temporary ultrasonic image is displayed. The temporary driving conditions mentioned here indicate driving conditions which are applied in a case where there is touch input assuming a case where there is no touch input is a normal case (driving conditions in this case are referred to as normal driving conditions) (step S5 in FIG. 6).

In a case where there is no touch input in step S1 (No in step S1), normal driving conditions are determined. In a case where the latest driving conditions are the temporary driving conditions, the driving controller 7 switches the temporary driving conditions to the normal driving conditions. If the latest driving conditions are the normal driving conditions, the driving controller 7 does not switch between the driving conditions (step S6 in FIG. 6).

In a case where there is no touch input, transmission and reception of ultrasonic waves are performed so that an ultrasonic image is displayed under the normal driving conditions (step S7 in FIG. 6).

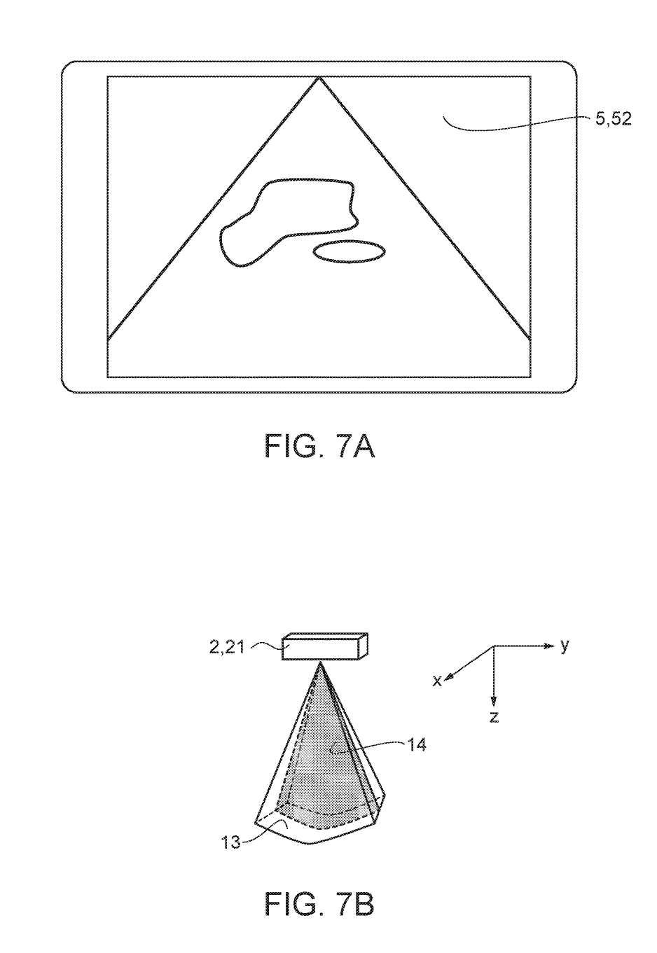

With reference to FIGS. 7A to 12B, a description will be made of the type of touch input and a driving control method corresponding thereto. Throughout FIGS. 7A to 12B, the drawings with the suffix A illustrate an ultrasonic image (a B mode image in the present embodiment) displayed on the display 5, and the drawings with the suffix B illustrate a relationship between the ultrasonic element array 21 and an ultrasonic beam scanning surface. The ultrasonic element array 21 has the central axis disposed in the z axis direction on the xy plane. An ultrasonic beam scanning range and an imaging range are indicated by the three-dimensional volume scanning range 13 or the two-dimensional scanning range 14 in sector scanning. A description will be made of real-time driving control of the two-dimensional scanning range 14 based on the user's touch input.

FIG. 7A is a diagram illustrating a B mode image on a driving scanning surface under initial driving conditions. FIG. 7B is a diagram for explaining the two-dimensional scanning range 14 corresponding to the B mode image in FIG. 7A. As illustrated in FIGS. 7A and 7B, the two-dimensional scanning range 14 is a plane which is parallel to the yz plane under the initial driving conditions.

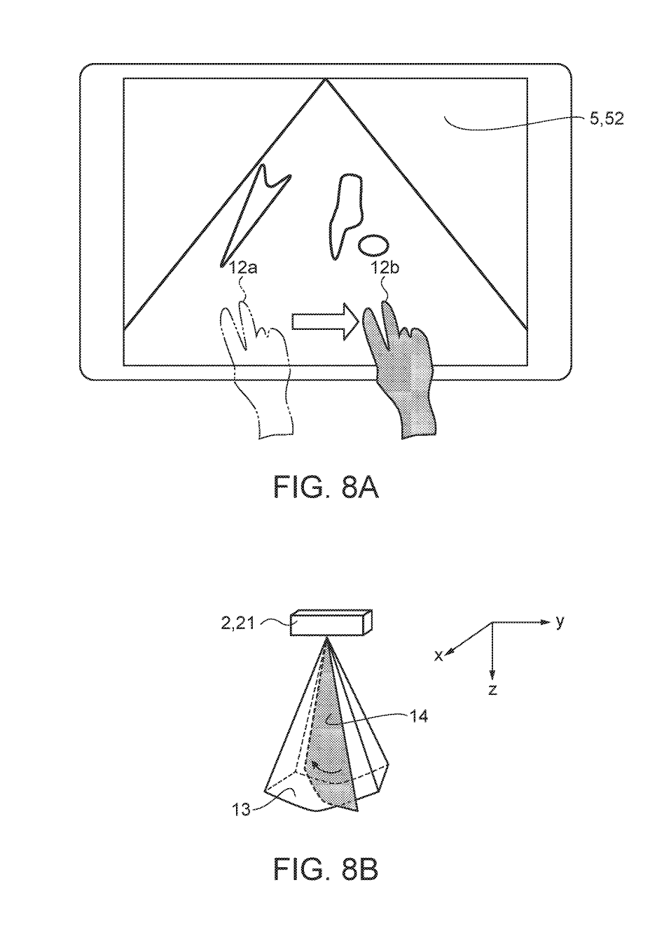

FIG. 8A is a diagram illustrating an operation on the touch panel and a B mode image when z-axis rotation driving control is performed. FIG. 8B is a diagram for explaining the two-dimensional scanning range 14 corresponding to the operation on the touch panel and the B mode image in FIG. 8A.

### CONTROL EXAMPLE 1

### z Axis Rotation of Two-Dimensional Scanning Range

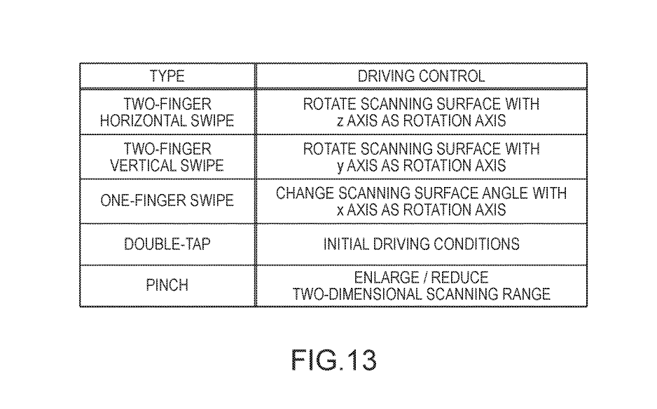

As illustrated in FIG. 8A, the user performs two-finger swipe from a touch position 12a to a touch position 12b on the touch panel 52 of the display 5. The touch input determinator 61 determines that there is touch input, and the touch position detector 62 detects the touch position 12a at a certain sampling time point t. The touch position detector 62 detects a touch position every sampling interval Δt, and detects the touch position 12b within a predetermined time t+n·Δt (where n is a real number). In a case of the two-finger operation, this touch operation is simultaneously detected at two adjacent positions. The touch operation determinator 6 determines that the type of touch operation is two-finger horizontal swipe on the basis of the detection result in the touch position detector 62. The touch operation determinator 6 calculates the operation velocity v and the movement amount d by using Equations (1) and (2). The term “horizontal” is the same as the horizontal direction on the drawing. The driving controller 7 receives the type of touch operation, the operation velocity, and the movement amount of the operation from the touch operation determinator 6. FIG. 13 is a diagram illustrating driving condition correspondence. The driving controller 7 selects predetermined driving conditions from the driving condition correspondence as illustrated in FIG. 13, and controls driving of the ultrasonic wave transmitter/receiver 22. For example, driving control for the two-finger horizontal swipe is rotation driving control (refer to the two-dimensional scanning range 14 in FIG. 8B) for the two-dimensional scanning range 14 with the z axis as a rotation axis. A rotation direction of the scanning range may be appropriately determined on the basis of a direction of horizontal swipe.

Hereinafter, it is assumed that correspondence between a touch operation and driving control is read from the driving control correspondence in FIG. 13.

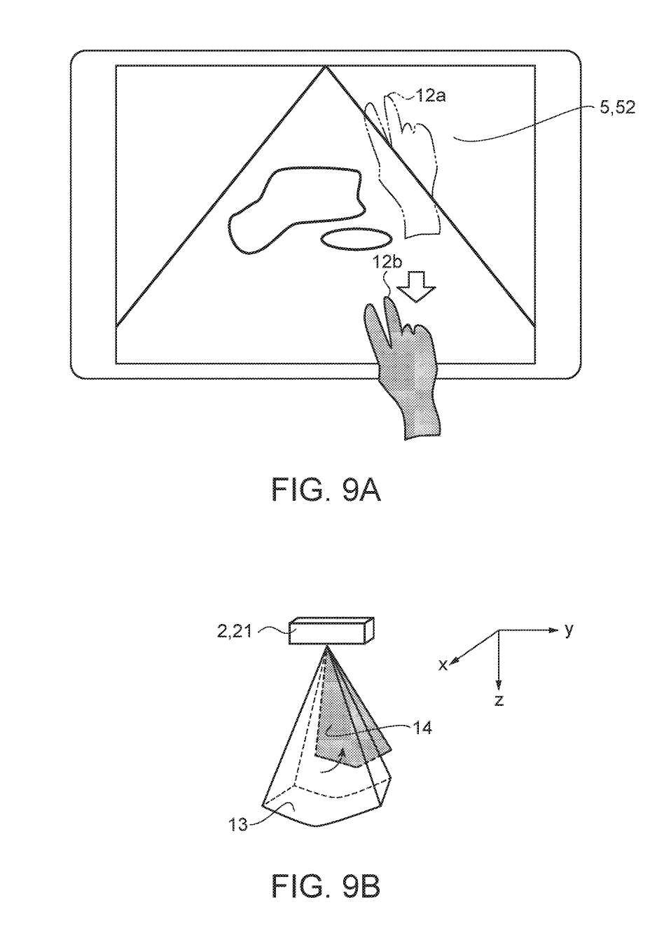

FIG. 9A is a diagram illustrating an operation on the touch panel and a B mode image when y-axis rotation driving control is performed. FIG. 9B is a diagram for explaining the two-dimensional scanning range 14 corresponding to the operation on the touch panel and the B mode image in FIG. 9A.

### CONTROL EXAMPLE 2

### y Axis Rotation of Two-Dimensional Scanning Range

As illustrated in FIG. 9A, the user performs two-finger swipe from a touch position 12a to a touch position 12b on the touch panel 52 of the display 5. In the same as in the control example 1, the touch operation determinator 6 determines that the type of touch operation is two-finger vertical swipe on the basis of the detection result in the touch position detector 62, and calculates the operation velocity v and the movement amount d. The term “horizontal” is the same as the vertical direction on the drawing. Driving control for the two-finger vertical swipe is rotation driving control (refer to the two-dimensional scanning range 14 in FIG. 9B) for the two-dimensional scanning range 14 with the y axis as a rotation axis. A rotation direction of the scanning range may be appropriately determined on the basis of a direction of vertical swipe.

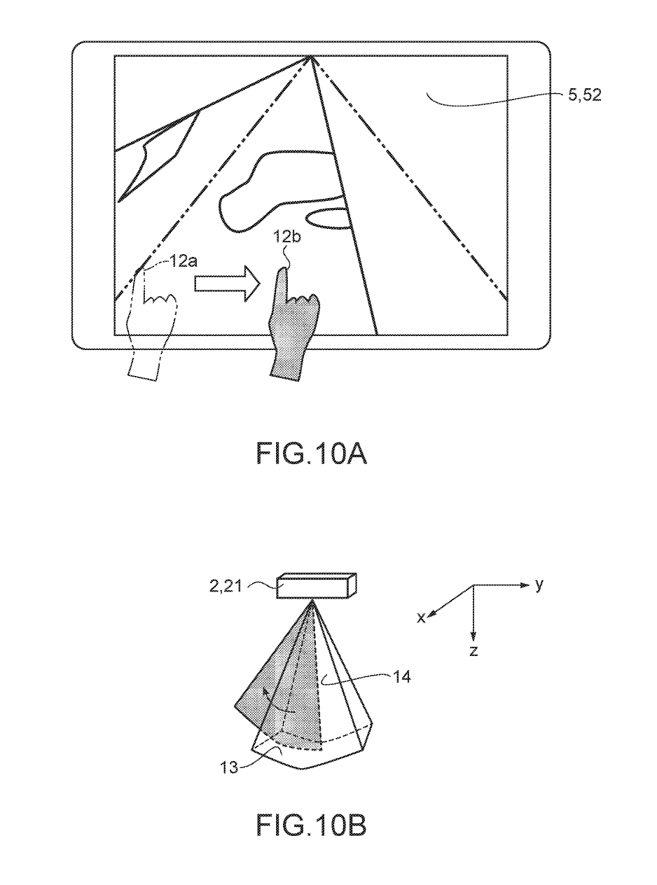

FIG. 10A is a diagram illustrating an operation on the touch panel and a B mode image when x-axis rotation driving control is performed. FIG. 10B is a diagram for explaining the two-dimensional scanning range 14 corresponding to the operation on the touch panel and the B mode image in FIG. 10A.

### CONTROL EXAMPLE 3

### x Axis Rotation of Two-Dimensional Scanning Range

As illustrated in FIG. 10A, the user performs one-finger swipe from a touch position 12a to a touch position 12b on the touch panel 52 of the display 5. In the same as in the control example 1, the touch operation determinator 6 determines that the type of touch operation is one-finger horizontal swipe on the basis of the detection result in the touch position detector 62, and calculates the operation velocity v and the movement amount d. Driving control for the one-finger horizontal swipe is rotation driving control (refer to the two-dimensional scanning range 14 in FIG. 10B) for the two-dimensional scanning range 14 with the x axis as a rotation axis. A two-dimensional scanning range during this rotation driving is located on the same plane before and after rotation, but this scanning is defined as a scanning surface angle change. A rotation direction of the scanning range may be appropriately determined on the basis of a direction of horizontal swipe.

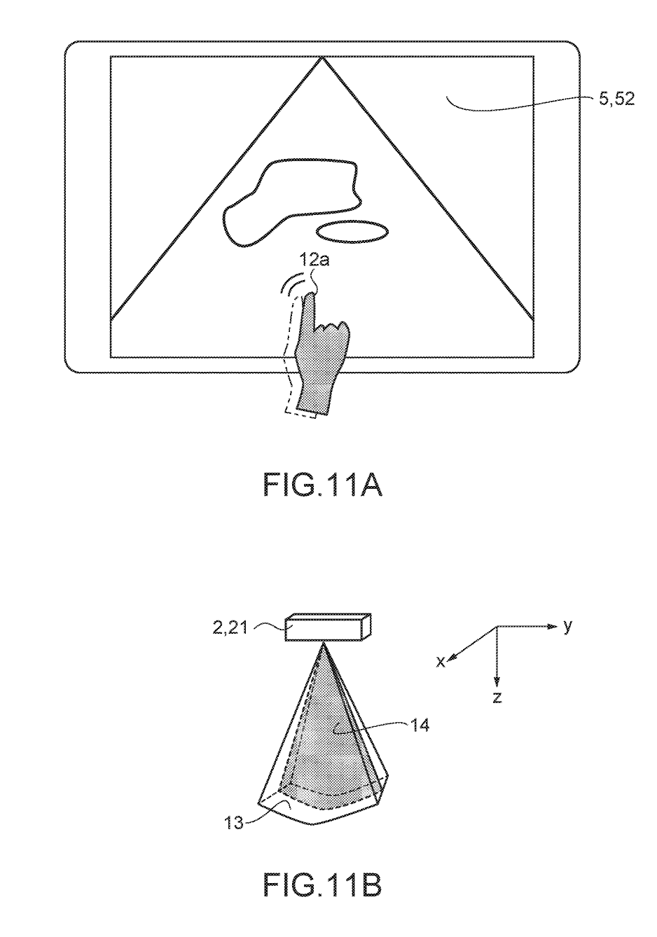

FIG. 11A is a diagram illustrating an operation on the touch panel and a B mode image when driving control is performed under initial driving conditions. FIG. 11B is a diagram for explaining the two-dimensional scanning range 14 corresponding to the operation on the touch panel and the B mode image in FIG. 11A.

### CONTROL EXAMPLE 4

### Initial Driving Condition Driving for Two-Dimensional Scanning Range

As illustrated in FIG. 11A, the user performs double-tap (continuously tapping the touch panel twice) at a touch position 12a on the touch panel 52 of the display 5. In the same as in the control example 1, the touch operation determinator 6 determines that the type of touch operation is double-tap on the basis of the detection result in the touch position detector 62. Driving control for the double-tap is control in which initial driving conditions stored in the storage 8 in advance are read, and initial driving is performed (refer to the two-dimensional scanning range 14 in FIG. 11B). The initial driving conditions are preferably set so that a central axis of a two-dimensional scanning range is parallel to the z axis. The user may set desired driving conditions in advance.

According to the control example 4, the driving controller 7 of the ultrasonic diagnosis apparatus 1 including the storage 8 in which the initial driving conditions are stored may driving control under the initial driving conditions on the basis of a touch operation which is input on the touch panel.

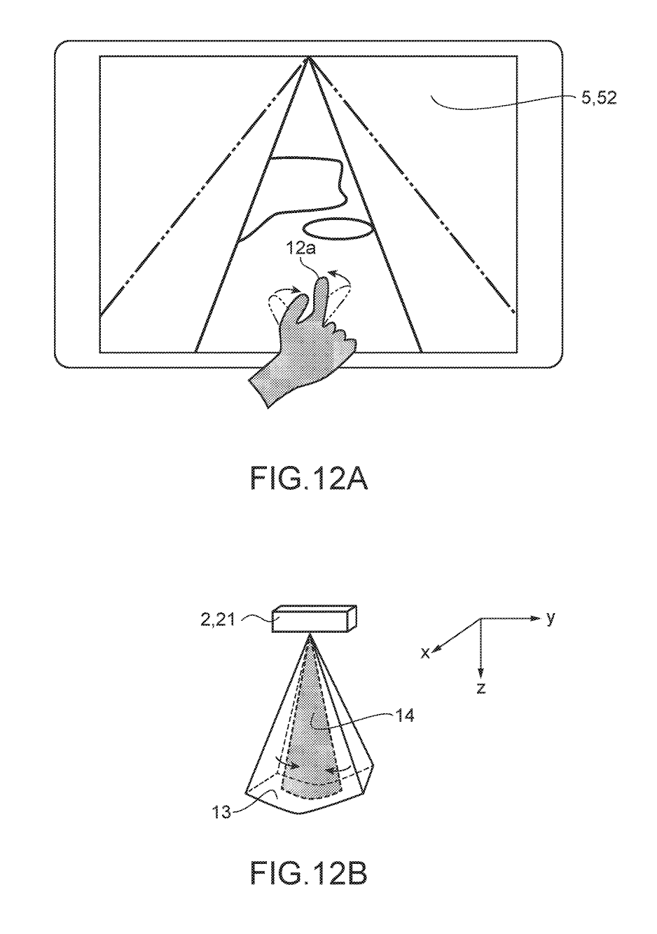

FIG. 12A is a diagram illustrating an operation on the touch panel and a B mode image when enlargement/reduction driving control is performed. FIG. 12B is a diagram for explaining the two-dimensional scanning range 14 corresponding to the operation on the touch panel and the B mode image in FIG. 12A.

### CONTROL EXAMPLE 5

### Enlargement/Reduction of Two-Dimensional Scanning Range

As illustrated in FIG. 12A, the user performs pinch (pinching or unpinching a screen with two fingers) at a touch position 12a on the touch panel 52 of the display 5. In the same as in the control example 1, the touch operation determinator 6 determines that the type of touch operation is a pinch operation on the basis of the detection result in the touch position detector 62, and calculates the operation velocity v and the movement amount d. Driving control for the pinch operation is scanning angle range control in the yz plane of the two-dimensional scanning range 14 based on sector scanning (refer to the two-dimensional scanning range 14 in FIG. 12B). In FIGS. 12A and 12B, a scanning angle is reduced through a pinching operation. Conversely, in a case of an unpinching operation, driving control is performed so that a scanning angle is increased.

As described in the control examples 1 to 5, the driving controller 7 of the ultrasonic diagnosis apparatus 1 can control either one of a driving scanning surface or a scanning surface angle in the ultrasonic element array 21 on the basis of the type of touch operation corresponding to an input signal from the touch panel.

Next, a description will be made of driving control based on a movement amount of a touch operation with reference to FIGS. 7A and 7B and FIGS. 14A to 17. Throughout FIGS. 7A and 7B and FIGS. 14A to 15B, the drawings with the suffix A illustrate an ultrasonic image (a B mode image in the present embodiment) displayed on the display 5, and the drawings with the suffix B illustrate a relationship between the ultrasonic element array 21 and an ultrasonic beam scanning surface. The ultrasonic element array 21 has the central axis disposed in the z axis direction on the xy plane. An ultrasonic beam scanning range and an imaging range are indicated by the three-dimensional volume scanning range 13 or the two-dimensional scanning range 14 in sector scanning. Herein, a description will be made of real-time driving control of the two-dimensional scanning range 14 based on a movement amount of a touch operation. A movement amount of a touch operation may be replaced with a movement amount per unit time, that is, the velocity of the touch operation.

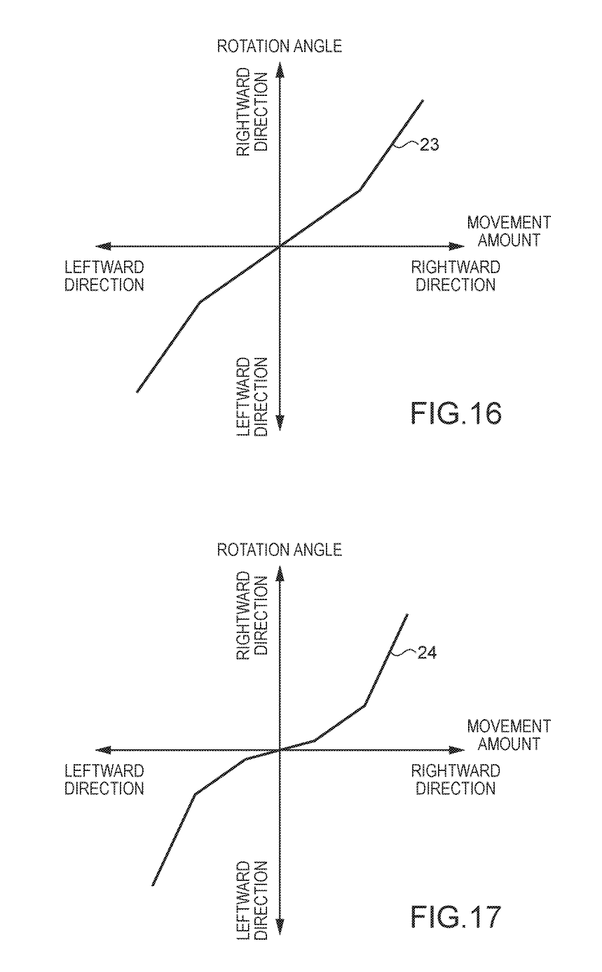

FIG. 16 is a diagram for explaining a relationship between a movement amount of a touch operation and a rotation angle of a scanning range. In FIG. 16, a transverse axis expresses a movement amount of swipe, and a longitudinal axis expresses a rotation angle of the two-dimensional scanning range 14. A first relationship line 23 indicates a relationship between a movement amount of swipe and a rotation angle of the two-dimensional scanning range 14. A rotation angle is set to be small in a range in which a movement amount is relatively small. A change of a rotation angle for a change of a movement amount is set to be small, that is, the inclination of the first relationship line 23 is set to be small. A rotation angle is set to be large in a range in which a movement amount is relatively large. A change of a rotation angle for a change of a movement amount is set to be large, that is, an inclination of the first relationship line 23 is set to be large. Consequently, if the user performs large swipe, the rotation of the two-dimensional scanning range 14 can be increased. Conversely, if the user performs large swipe, the rotation of the two-dimensional scanning range 14 can be reduced. A movement amount may be regarded as a movement amount per unit time, and a rotation angle of a scanning range may be set according to the velocity of an operation.

FIG. 17 is a diagram for explaining a relationship between a movement amount of a touch operation and a rotation angle of a scanning range. In FIG. 17, a transverse axis expresses a movement amount of a touch operation, and a longitudinal axis expresses a rotation angle of the two-dimensional scanning range 14. A second relationship line 24 indicates a relationship between a movement amount of swipe and a rotation angle of the two-dimensional scanning range 14. A change of a rotation angle of a scanning range for a movement amount of a touch operation may be set to three stages as in the second relationship line 24, or multiple stages equal to or higher than that. In this case, as a movement amount is increased, the inclination of the second relationship line 24 is preferably set to be increased.

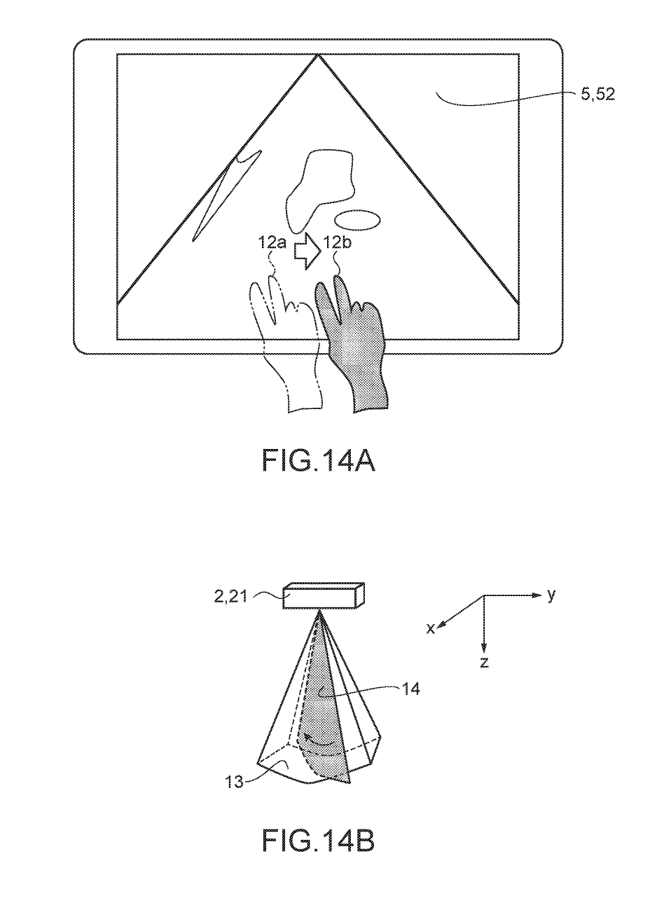

FIG. 14A is a diagram illustrating an operation on the touch panel and a B mode image when rotation angle control is performed. FIG. 14B is a diagram for explaining the two-dimensional scanning range 14 corresponding to the operation on the touch panel and the B mode image in FIG. 14A. The user performs two-finger swipe from a touch position 12a to a touch position 12b illustrated in FIG. 14A on the touch panel 52 of the display 5. According to the control example 1, the touch operation determinator 6 determines that the type of touch operation is two-finger rightward swipe on the screen on the basis of the detection result in the touch position detector 62, and calculates the operation velocity v and the movement amount d. The driving controller 7 receives the type of touch operation, the operation velocity, and the movement amount of the operation from the touch operation determinator 6. In this case, a value of a rotation angle to be controlled for driving is calculated on the basis of a relationship between a movement amount and a rotation angle as illustrated in FIG. 16. As illustrated in FIG. 14B, control for rotation driving of the two-dimensional scanning range 14 with the z axis as a rotation axis is performed.

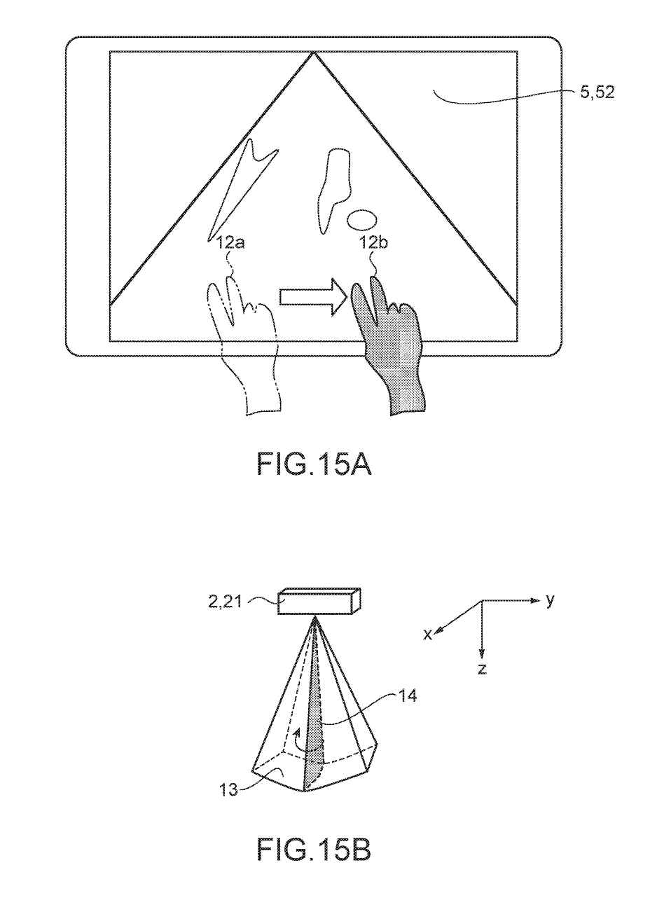

FIG. 15A is a diagram illustrating an operation on the touch panel and a B mode image when rotation angle control is performed with respect to a case where a movement amount of a touch operation is larger than that in FIG. 14A. FIG. 15B is a diagram for explaining the two-dimensional scanning range 14 corresponding to the operation on the touch panel and the B mode image in FIG. 15A. In this case, since a rotation angle larger than in the case illustrated in FIG. 14A is set, and rotation driving is controlled, as illustrated in FIG. 15B, larger rotation of the two-dimensional scanning range 14 is performed in FIG. 15B than in FIG. 14B.

As mentioned above, the driving controller 7 of the ultrasonic diagnosis apparatus 1 can perform driving control according to any of the type of touch operation, the velocity of the operation, and a movement amount of the operation which are input from the touch panel.

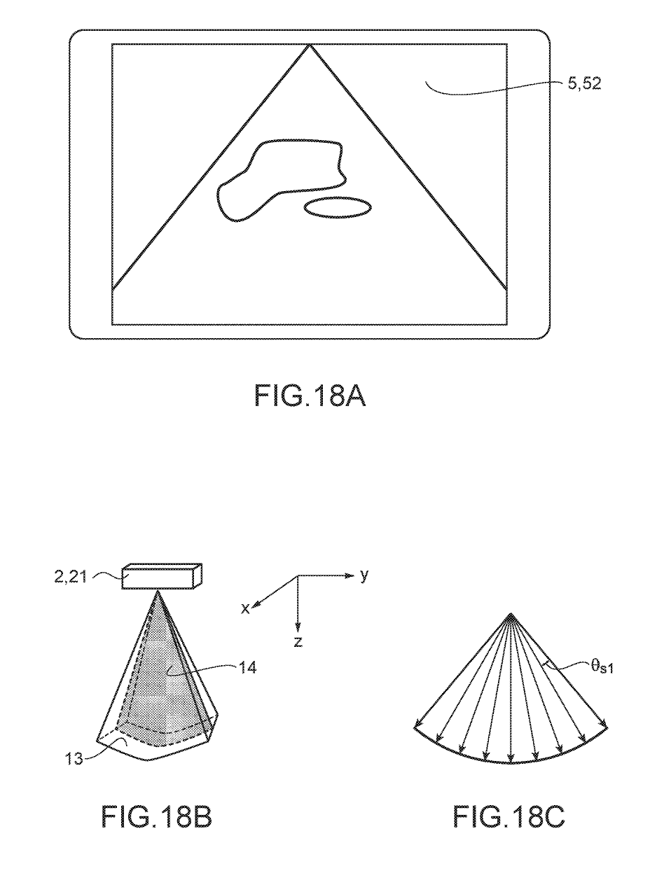

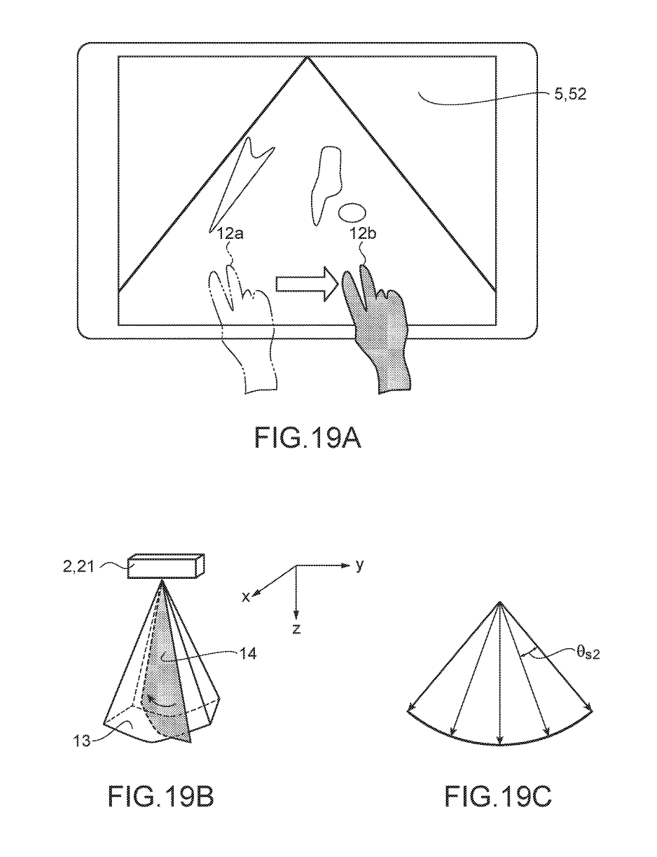

With reference to FIGS. 18A to 19C, a description will be made of a driving control method in cases where there is input and there is no input from the touch panel. Throughout FIGS. 18A to 19C, the drawings with the suffix A illustrate an ultrasonic image (a B mode image in the present embodiment) displayed on the display 5, the drawings with the suffix B illustrate a relationship between the ultrasonic element array 21 and an ultrasonic beam scanning surface, and the drawings with the suffix C are schematic diagrams illustrating ultrasonic beams in a scanning range as arrows. The ultrasonic element array 21 has the central axis disposed in the z axis direction on the xy plane. An ultrasonic beam scanning range and an imaging range are indicated by the three-dimensional volume scanning range 13 or the two-dimensional scanning range 14 in sector scanning.

FIG. 18A is a diagram illustrating a B mode image on a driving scanning surface under initial driving conditions. FIG. 18B is a diagram for explaining the two-dimensional scanning range 14 corresponding to the B mode image in FIG. 18A. FIG. 18C is a schematic diagram illustrating ultrasonic beams in the scanning range in FIG. 18B as arrows. As illustrated in FIG. 18A, a B mode image is displayed under initial driving conditions. As illustrated in FIG. 18B, the two-dimensional scanning range 14 is set under the initial driving conditions. The ultrasonic probe 2 actually transmits and receives an ultrasonic beam for each scanning angle step θs1 as a scanning line interval as illustrated in FIG. 18C. This corresponds to a case where there is no touch input, driving conditions in this case are referred to as normal driving conditions, and a mode in this case is set to a high resolution mode.

### CONTROL EXAMPLE 6

### Low Resolution Mode

FIG. 19A is a diagram illustrating a B mode image obtained through an operation on the touch panel in a low resolution mode. FIG. 19B is a diagram for explaining the two-dimensional scanning range 14 corresponding to the B mode image in FIG. 19A. FIG. 19C is a schematic diagram illustrating ultrasonic beams in the scanning range in FIG. 19B as arrows. As illustrated in FIG. 19A, the user performs two-finger swipe from a touch position 12a to a touch position 12b on the touch panel 52 of the display 5. In order to perform temporary driving control while there is touch input, the driving controller 7 sets a scanning angle step θs2 as in FIG. 19C to be larger than the scanning angle step θs1 (θs1<θs2). The number of ultrasonic beams corresponding to the number of scanning lines is reduced, and driving control is performed so that driving is performed in the same scanning range as in normal driving. Since the number of ultrasonic beams is reduced, and thus the time required to transmit and receive ultrasonic waves for each frame can be reduced, a frame rate of generating ultrasonic images can be improved. Thus, it is possible to reduce screen display delay during a swipe operation. As illustrated in FIG. 19B, the ultrasonic probe 2 changes driving conditions so as to control driving of an ultrasonic beam.

Regarding temporary driving control, parallel simultaneous reception control may be performed. The parallel simultaneous reception control is a method in which a range of transmitted ultrasonic beams is increased, and a plurality of received waves are acquired within the range of the beams, and the number of transmitted beams can be reduced. The direction of an ultrasonic beam is reduced, and thus an azimuth resolution is reduced, but a frame rate can be improved with respect to the normal driving conditions. Thus, it is possible to reduce screen display delay during a swipe operation.

As mentioned above, the driving controller 7 of the ultrasonic diagnosis apparatus 1 is driven in a high resolution mode in a case where there is no input from the touch panel, and is driven in a low resolution mode in a case where there is input from the touch panel. The number of scanning lines can be reduced or a scanning line interval can be increased in the low resolution mode more than in the high resolution mode.

As described above, according to the ultrasonic diagnosis apparatus 1 according to the present embodiment, the following effects can be achieved.

### Effect 1

According to the present embodiment, the ultrasonic element array 21 having two-dimensional arrangement transmits ultrasonic waves and receives reflected echoes. The ultrasonic image generator 4 generates an ultrasonic image on the basis of the received reflected echoes. The display 5 displays the generated ultrasonic image. A user performs a touch operation on the touch panel 52 provided on the display surface of the display 5. The driving controller 7 which controls driving of the ultrasonic element array 21 controls driving of the ultrasonic element array 21 on the basis of an input signal from the touch panel due to a touch operation. The touch operation is easier than a keyboard operation or a track ball operation. Therefore, the user can easily perform real-time driving control based on the user's touch operation.

### Effect 2

According to the present embodiment, the driving controller 7 changes driving control according to the type of touch operation, the velocity of the operation, and a movement amount of the operation which are input from the touch panel 52. Thus, a user can easily change driving control for the ultrasonic element array 21.

### Effect 3

According to the present embodiment, the driving controller 7 can easily switch between observation regions by controlling a driving scanning surface in the ultrasonic element array 21 having two-dimensional arrangement. Alternatively, it is possible to easily switch between observation angles by controlling a scanning surface angle.

### Effect 4

According to the present embodiment, the driving controller 7 performs driving control on the basis of the initial driving conditions stored in advance and input from the touch panel. Thus, a user can easily execute the initial driving conditions even after driving conditions are variously changed.

### Effect 5

According to the present embodiment, the driving controller 7 controls driving of the ultrasonic element array 21 in the high resolution mode in a case where there is no input from the touch panel. The driving controller 7 controls driving of the ultrasonic element array 21 in the low resolution mode in a case where there is input from the touch panel. The number of scanning lines is reduced or a scanning line interval is increased in the low resolution mode more than in the high resolution mode. Thus, in a case where there is no input from the touch panel 52, a user can observe a high-resolution image in the high resolution mode. In a case where there is input from the touch panel 52, the user can observe an image in the low resolution mode without image display delay.

The invention is not limited to the above-described embodiment, and the above-described embodiment may be variously modified or altered. Modification examples will be described below.

### MODIFICATION EXAMPLE 1

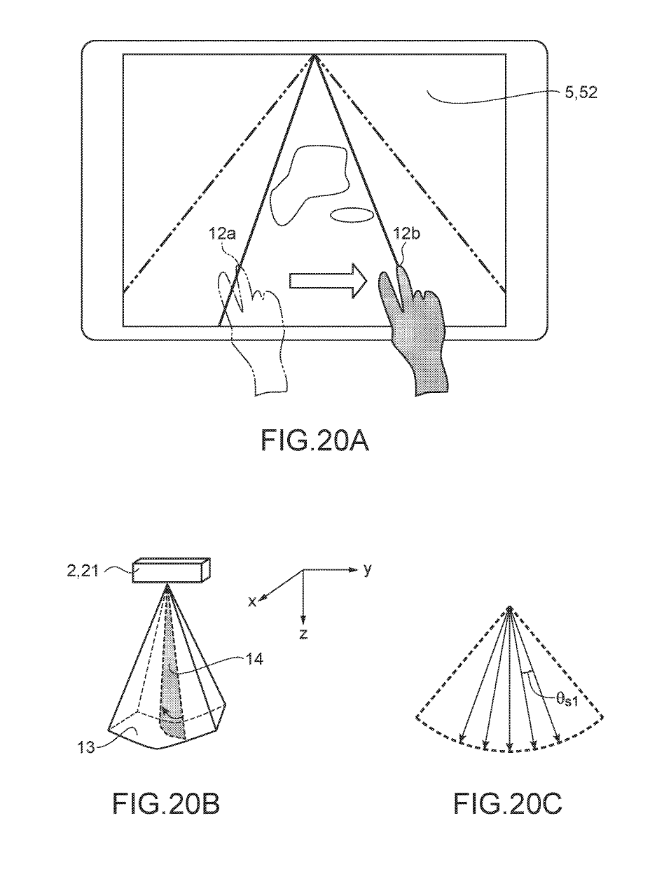

FIG. 20A is a diagram illustrating a B mode image obtained through an operation on the touch panel in a narrow visual field mode. FIG. 20B is a diagram for explaining the two-dimensional scanning range 14 corresponding to the B mode image in FIG. 20A. FIG. 20C is a schematic diagram illustrating ultrasonic beams in the scanning range in FIG. 20B as arrows. With reference to FIGS. 20A to 20C, a description will be made of a modification example in temporary driving condition setting. A region desired to be observed by a user is normally located at the screen center. As illustrated in FIG. 20A, the user performs a swipe operation from a touch position 12a indicating a region desired to be observed to a touch position 12b. As illustrated in FIG. 20B, the driving controller 7 performs driving control so that ultrasonic waves are transmitted and received in only a screen center range under temporary driving conditions (narrow visual field mode). As illustrated in FIG. 20C, since the number of ultrasonic beams is reduced more than in the normal driving conditions in FIG. 18C, and thus the time required to transmit and receive ultrasonic waves for each frame can be reduced, a frame rate of generating ultrasonic images can be improved. Thus, it is possible to reduce screen display delay during a swipe operation. Consequently, the low resolution mode may be replaced with the narrow visual field mode.

According to the present modification example, the driving controller 7 controls driving of the ultrasonic element array 21 in the high resolution mode in a case where there is no input from the touch panel. The driving controller 7 controls driving of the ultrasonic element array 21 in the low resolution mode in a case where there is input from the touch panel. The number of scanning lines is reduced or a scanning line interval is increased in the low resolution mode more than in the high resolution mode. Thus, in a case where there is no input from the touch panel 52, a user can observe a high-resolution image in the high resolution mode. In a case where there is input from the touch panel 52, the user can observe an image in the low resolution mode without image display delay.

### MODIFICATION EXAMPLE 2

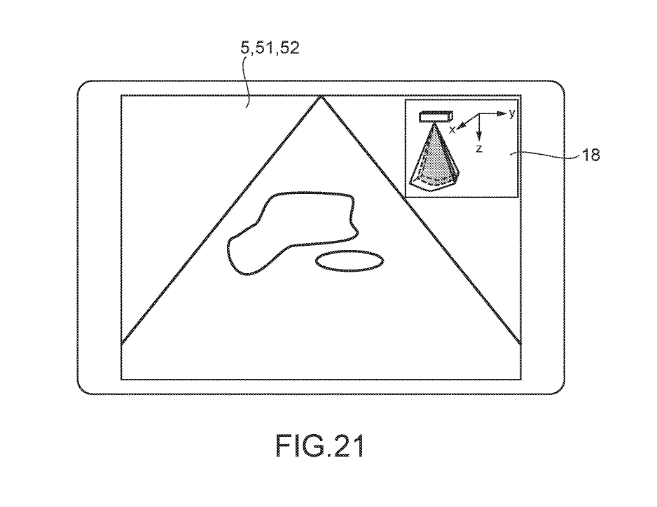

FIG. 21 is a diagram for explaining that a screen regarding a relationship among a B mode image in the two-dimensional scanning range 14, the ultrasonic element array 21, and an ultrasonic beam scanning surface is displayed on the monitor 51. If driving conditions are set in step S4 or step S6, the driving controller 7 stores the driving conditions in the storage 8 or a memory (not illustrated). In step S5 or step S7, the display 5 acquires ultrasonic image data from the ultrasonic image generator 4 and also acquires the driving conditions at a corresponding time phase from the storage 8 or the memory (not illustrated). As illustrated in FIG. 21, the display 5 displays screens such as FIGS. 18A to 18C illustrating a relationship between the ultrasonic element array 21 and an ultrasonic beam scanning surface, estimated from the driving conditions, on the monitor 51 along with the ultrasonic image data.

According to the present modification example, the display 5 displays screens such as FIGS. 18A to 18C illustrating a relationship among a B mode image, the ultrasonic element array 21, and an ultrasonic beam scanning surface together, and thus a user can easily recognize a relationship between a touch operation and driving control.

This application claims the benefit of foreign priority to Japanese Patent Application No. JP 2016-212647, filed Oct. 31, 2016, which is incorporated by reference in its entirety.

What is claimed is:

1. An ultrasonic diagnosis apparatus comprising:
an ultrasonic element array that has two-dimensionally arranged ultrasonic elements transmitting ultrasonic waves and receiving reflected echoes;
an ultrasonic image generator that generates an ultrasonic image on the basis of the reflected echoes received by the ultrasonic element array;
a display that displays the ultrasonic image;
a touch panel that is provided on a display surface of the display and receives a user's input so as to output an input signal; and
a driving controller that controls driving of the ultrasonic element array on the basis of the input signal from the touch panel.

2. The ultrasonic diagnosis apparatus according to claim 1,
wherein the driving controller performs driving control according to any of the type of touch operation, the velocity of the operation, and a movement amount of the operation which are input from the touch panel.

3. The ultrasonic diagnosis apparatus according to claim 1,
wherein the driving controller controls either one of a driving scanning surface and a scanning surface angle in the ultrasonic element array on the basis of the input signal from the touch panel.

4. The ultrasonic diagnosis apparatus according to claim 1, further comprising:
a storage that stores initial driving conditions,
wherein the driving controller performs driving control under the initial driving conditions on the basis of input from the touch panel.

5. The ultrasonic diagnosis apparatus according to claim 1,
wherein the driving controller performs driving in a high resolution mode in a case where there is no input from the touch panel, and performs driving in a low resolution mode in a case where there is input from the touch panel, and
wherein the driving is performed so that the number of scanning lines is reduced or a scanning line interval is increased in the low resolution mode more than in the high resolution mode.
